(19) United States
(12) Patent Application Publication
(10) Pub. No.: US 2015/0248945 A1
(43) Pub. Date: Sep. 3, 2015

(54) APPARATUS TO EMIT THERAPEUTIC RADIATIONS

(71) Applicant: ASA SRL, Arcugnano (IT)

(72) Inventors: Roberto Marchesini, Schio (IT); Lucio Zaghetto, Camposanpietro (IT)

(73) Assignee: ASA SRL, Arcugnano (IT)

(21) Appl. No.: 14/430,003

(22) PCT Filed: Sep. 13, 2013
(86) PCT No.: PCT/IB2013/002009

§ 371 Date: Mar. 20, 2015

(30) Foreign Application Priority Data
Sep. 20, 2012 (IT) UD2012A000161

(51) Int. Cl. G21K 5/10; A61N 5/02; A61N 2/00; A61N 5/01; A61N 5/06; A61N 5/10

(52) U.S. Cl. CPC G21K 5/10; A61N 5/06; A61N 5/10; A61N 2/00; A61N 5/01; A61N 5/02; A61N 5/1081; A61N 2005/0651; A61N 2005/067

(57) ABSTRACT

Apparatus to emit therapeutic radiations comprising at least an emitter of radiations configured to emit radiations toward a base plane containing an axis of displacement, movement means configured to selectively move said emitter of radiations both along a second path, arched and lying on a first lying plane passing through the axis of displacement, and also along a third path arched and lying on a second lying plane passing through an axis of rotation, perpendicular to the axis of displacement. The movement means are configured to move the emitter of radiations also along a first path substantially rectilinear and parallel to the axis of displacement, so as to move the emitter of radiations parallel to the base plane.

### FIELD OF THE INVENTION

The present invention concerns an apparatus to emit therapeutic radiations, which can be applied in the medical and physio-therapeutic fields, the field of sport medicine and in all those applications where radiations are used with a therapeutic, curative, rehabilitative, aesthetic or surgical purpose. The source of therapeutic radiations can be of any type, such as laser, a LED source, a pulsed light, radio waves, magnetic fields or other.

### BACKGROUND OF THE INVENTION

In the state of the art concerning localized radiation emissions, such as x-rays or those emitted by a laser, various apparatuses are known, able to direct or converge the radiations emitted, in a selective and programmed manner, toward a determined point or part of a patient, whether human or animal.

In particular, from the Italian patent application for industrial invention UD2011A000045 filed in the name of the Applicant on 23 Mar. 2012, which is incorporated here for reference, an apparatus is known to emit therapeutic radiations, for example, but not only, radiations emitted by a laser, which comprises at least an emitter of radiations, which can be selectively translated along a rectilinear longitudinal axis and/or a transverse axis, which is also rectilinear but perpendicular to the longitudinal axis, and also moved along arched routes each lying on a lying plane passing through the longitudinal and/or transverse axis.

This known apparatus, even though it can guarantee a multiplicity of movements and high performance in many applications, is rather complex and costly.

EP 1.419.799 describes an apparatus for radiological treatment in which an irradiation head can move angularly along a curvilinear support which extends transversely with respect to the position of the patient, it can oscillate with respect to its pivoting point on said support, and can be raised and lowered vertically with respect to the position of the patient.

One purpose of the present invention is to obtain an apparatus to emit therapeutic radiations which, as well as being able to emit therapeutic radiations in an automated and programmable manner, in a direction substantially perpendicular to the surface of the patient to be treated, is simple to make and to use, as well as reliable and economical.

Another purpose is to obtain an apparatus to emit therapeutic radiations which guarantees in every operating condition a uniform, constant and efficient result of the treatment.

The Applicant has devised, tested and embodied the present invention to overcome the shortcomings of the state of the art and to obtain these and other purposes and advantages.

### SUMMARY OF THE INVENTION

The present invention is set forth and characterized in the independent claim, while the dependent claims describe other characteristics of the invention or variants to the main inventive idea.

In accordance with the above purposes, an apparatus to emit therapeutic radiations according to the present invention comprises at least an emitter of radiations, for example a laser, and movement means configured to selectively move said emitter of radiations both along a first substantially rectilinear path, parallel to an axis of displacement, and along a second path, substantially arched and lying on a first lying plane passing through the axis of displacement, and also along a third path, substantially arched and lying on a second lying plane passing through an axis of rotation, perpendicular to the axis of displacement, so that the axis of emission of the radiations is convergent toward a common point or isocenter, at the intersection of the axis of displacement with the axis of rotation.

The movement means have been studied and configured to allow the emitter of radiations to maintain the perpendicularity of the beam of radiation on the surface of the patient to be covered by the scanning. The surfaces involved can be nearly equivalent to spherical or hemispherical surfaces and/or cylindrical surfaces and/or flat surfaces.

In accordance with one characteristic of the present invention, the movement means essentially comprise: first guide means, substantially arched in shape, configured to allow the emitter of radiations to move along the third path; support means configured to support the first guide means so that they can rotate around the axis of rotation; and second guide means, configured to slidingly support the support means and to allow the support means, the first guide means and the emitter of radiations to move together along the first path. The movement means are configured so that the axis of emission of the radiations can always be directed toward the isocenter, irrespective of the position in which the emitter of radiations is located along any of the three paths.

Advantageously, at least one of the, or both arched paths are the arc of a circle with the common point as the center.

In accordance with another characteristic of the present invention, the movement means comprise a support slider, able to slide along the first guide means, with a substantially arched shape; moreover, the emitter of radiations is pivoted on the support slider and is commanded by an adjustment device able to selectively maintain the axis of emission of the radiations perpendicular to a base plane on which the isocenter and the axes of displacement and rotation lie.

In this way, by keeping the perpendicularity between the axis of emission of the radiations and the base plane constant and by driving the adjustment device while the emitter of radiations is selectively moved along the first and third path, it is possible to cover the scanning of the base plane.

According to another characteristic of the present invention, each movement of the emitter of radiations along any one whatsoever of the three paths is commanded by at least an electric motor, by means of mechanical members which guarantee that the motion is irreversible, thus preventing unwanted and/or unforeseeable external actions from modifying the position of the emitter of radiations.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a perspective view of an apparatus to emit therapeutic radiations according to the present invention in an initial position;

FIG. 2 is a perspective view of the apparatus in FIG. 1, in an operating position;

FIG. 3 is a perspective view of a first detail of the apparatus in FIG. 1, showing an arched guide for an emitter of radiations;

FIG. 4 is a perspective view of a second detail of the apparatus in FIG. 1, showing a support slider able to slide on the arched guide of FIG. 3;

FIG. 5 is another perspective view of the second detail of the apparatus in FIG. 1, showing the adjustment device mounted on the support slider of FIG. 4;

FIG. 6 is a perspective view of a third detail of the apparatus in FIG. 1, showing an arched support on which the arched guide of FIG. 3 is pivoted;

FIG. 7 is a perspective view showing an enlarged detail of a pivoting zone of the arched guide of FIG. 3 in the arched support of FIG. 6;

FIG. 8 is a perspective view of a fourth detail of the apparatus in FIG. 1, showing a rectilinear guide on which the arched support of FIG. 6 is able to slide;

FIGS. 9 and 10 respectively show a variant of FIG. 1 and FIG. 3;

FIGS. 11 and 12 show two possible applications of the present invention.

### DESCRIPTION OF ONE FORM OF EMBODIMENT OF THE PRESENT INVENTION

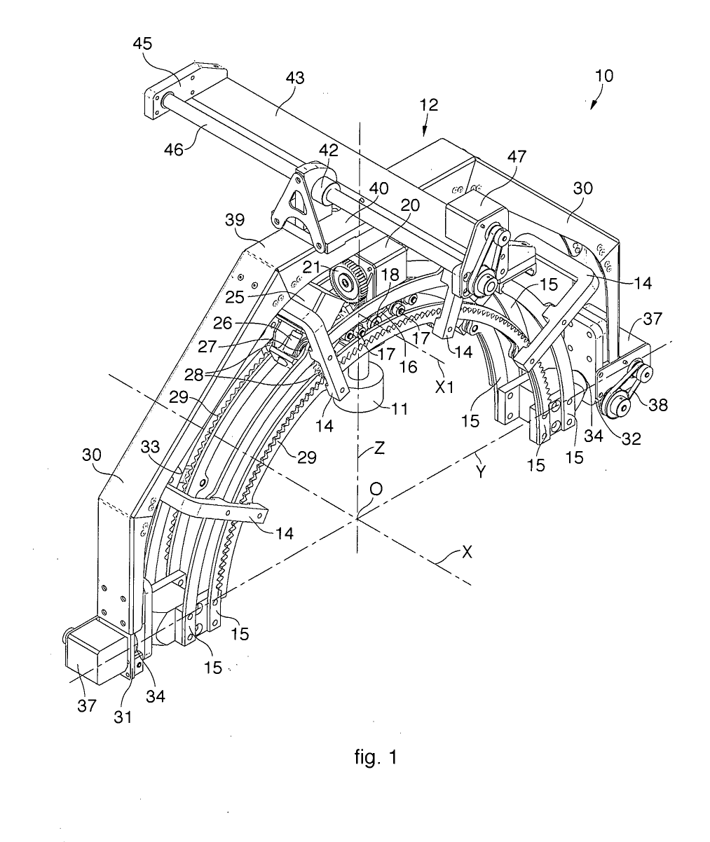

With reference to FIG. 1, an apparatus 10 to emit therapeutic radiations according to the present invention comprises an emitter of radiations 11, of a known type, for example a laser, which is associated to movement means 12, which will be described in detail hereafter and which are configured to selectively move the emitter of radiations 11 both along a first path P1 (FIG. 2), substantially rectilinear and parallel to an axis of displacement X, and along a second path P2, substantially shaped as the arc of a circle and lying on a first lying plane PG1 passing through the axis of displacement X, and along a third path (P3), also substantially shaped as the arc of a circle and lying on a second lying plane PG2 passing through an axis of rotation Y, perpendicular to the axis of displacement X, so that the axis of emission Z of the emitter of radiations 11 is convergent toward a common point or isocenter O, located at the intersection of the axis of displacement X with the axis of rotation Y. The isocenter O lies on a base plane PB, horizontal for example.

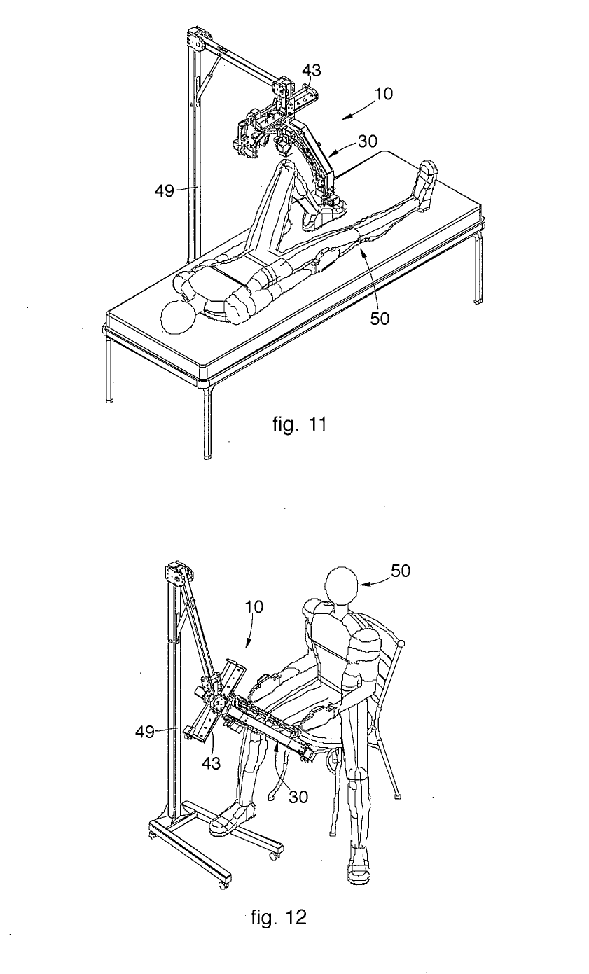

The base plane PB, which contains the axis of displacement X, identifies, in the description and in the drawings, the normal position of a patient 50 (FIG. 11), or of an area of the patient's body toward which the therapeutic radiations or other are made to converge (FIG. 12), toward which the emitter of radiations 11 has to be directed in order to carry out the desired therapeutic treatment.

The movement means 12 comprise an arched guide 13 which allows the emitter of radiations 11 to move along the third path P3 substantially transverse with respect to the axis of displacement X.

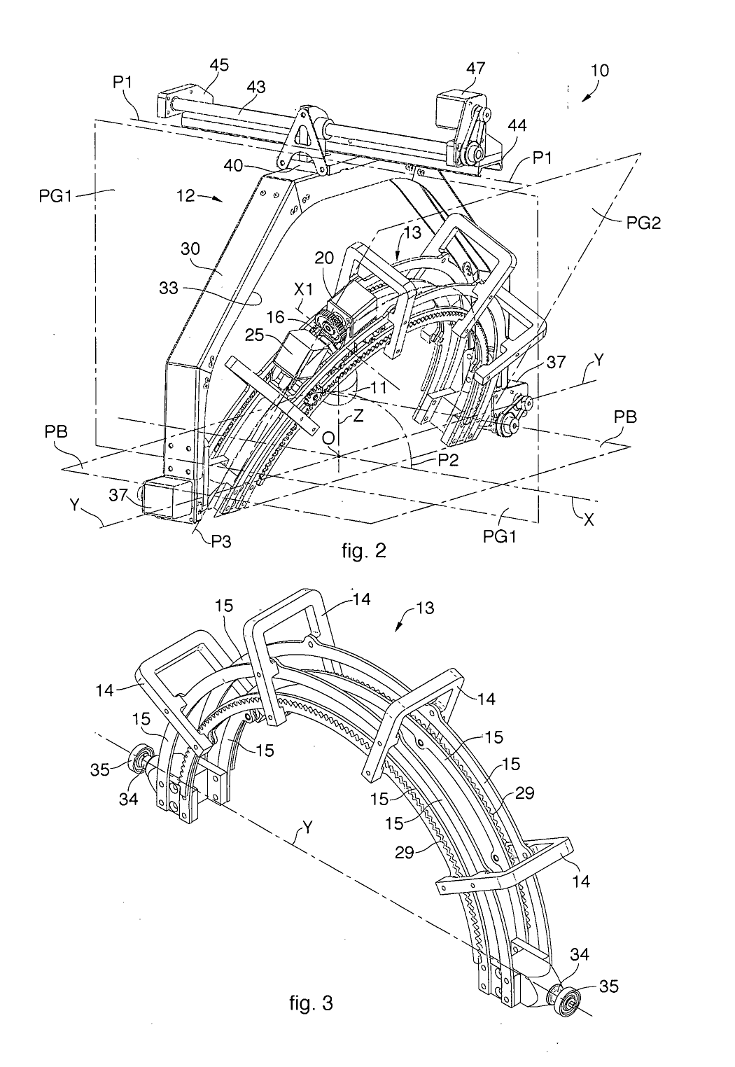

The arched guide 13 comprises four supports 14 (FIG. 3), five in the variant in FIG. 10, which are shaped like an upside down U, and support two pairs of arched tracks 15, parallel with respect to each other and with constant radii, all concentric to the isocenter O. The arched tracks 15 have a development of a little more than 180°, so that the emitter of radiations 11 can complete a travel of 180° along the third path P3.

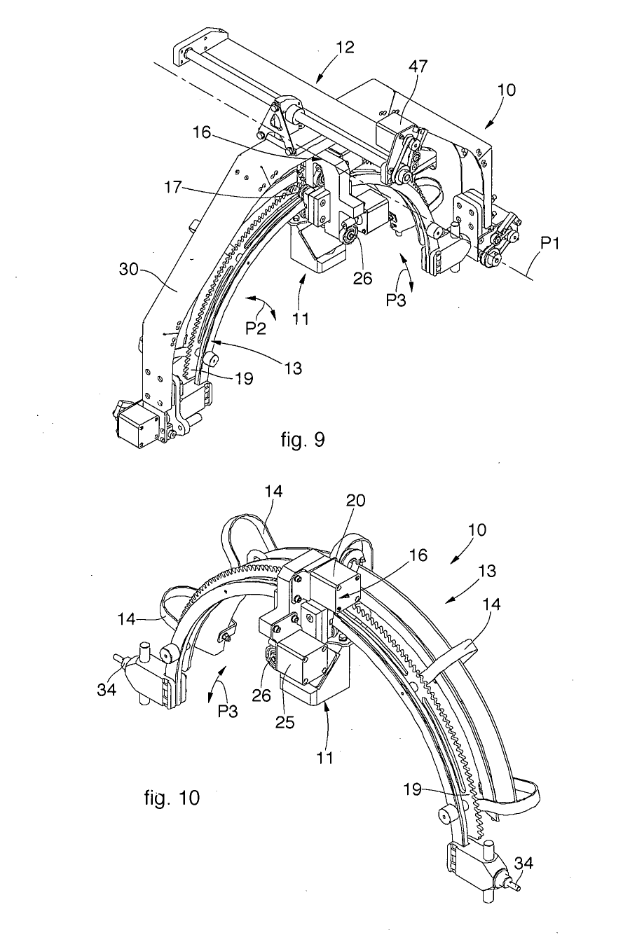

The variant shown in FIGS. 9 and 10 provides a single arched track 19.

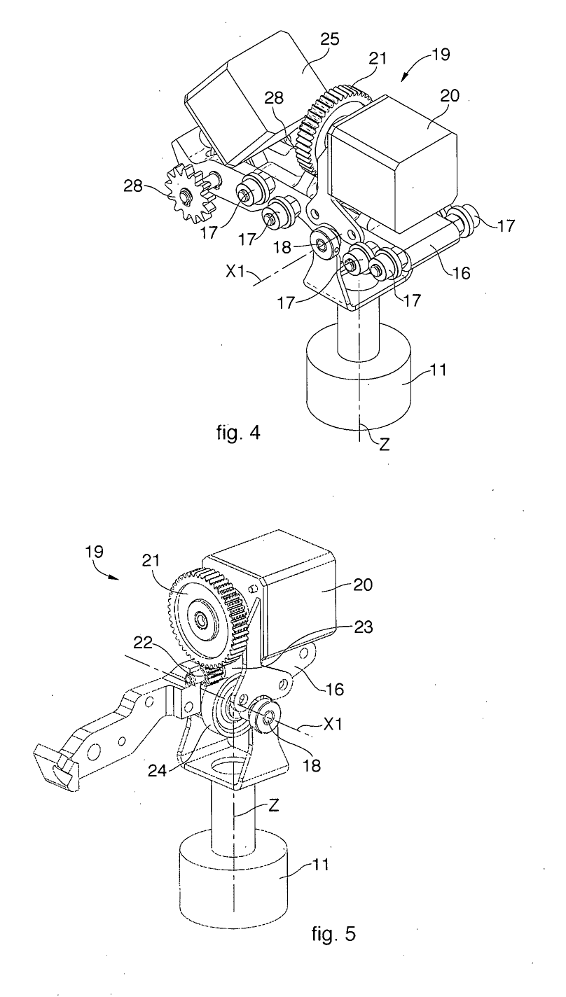

The emitter of radiations 11 is mounted on a support slider 16 (FIGS. 4 and 5) which is able to slide, using its wheels 17, on the arched tracks 15.

In particular, on each lateral flank of the support slider 16 four wheels 17 are mounted rotatable, and are disposed so that two of them are in contact with a corresponding lower arched track 15, while the other two are in contact with a corresponding upper arched track 15. In this way, the support slider 16 is guided with great precision on the arched tracks 15 and any involuntary movement thereof with respect to the arched guide 13 is prevented, without any risk of sagging or jolting. This result is also obtained even when the arched guide 13 is made to incline or oscillate around the axis of rotation Y, along the path P2.

In this case, the emitter of radiations 11 is mounted on a pin 18 pivoted on the support slider 18 and rotatable around an axis X1 (FIGS. 1, 2, 4 and 5).

The emitter of radiations 11 is connected to an adjustment device 19 mounted on the support slider 16 and suitable to adjust the perpendicularity, that is, the inclination of the axis of emission Z.

The adjustment device 19 (FIG. 5) comprises a first electric motor 20 suitable to make a toothed wheel 24, solid to the pin 18, rotate by means of two toothed wheels 21 and 22 and a worm screw 23. The connection between the worm screw 23 and the toothed wheel 24 is the irreversible type, so that the inclination of the axis of emission Z of the emitter of radiations 11 can be adjusted only by driving the electric motor 20.

The motion of the support slider 16 along the path P3 is achieved using a second electric motor 25 (FIG. 4), mounted on board the support slider 16 itself, and connected by means of a worm screw 26 (FIG. 1) and a toothed wheel 27 to a pair of toothed wheels 28 coaxial with respect to each other, which are constantly engaged with two corresponding arched racks 29, disposed below the arched tracks 15 and concentric to the isocenter O.

The connection between the worm screw 26 and the toothed wheel 27 is also the irreversible type, so the support slider 16 can move along the arched tracks 15 only by driving the electric motor 25.

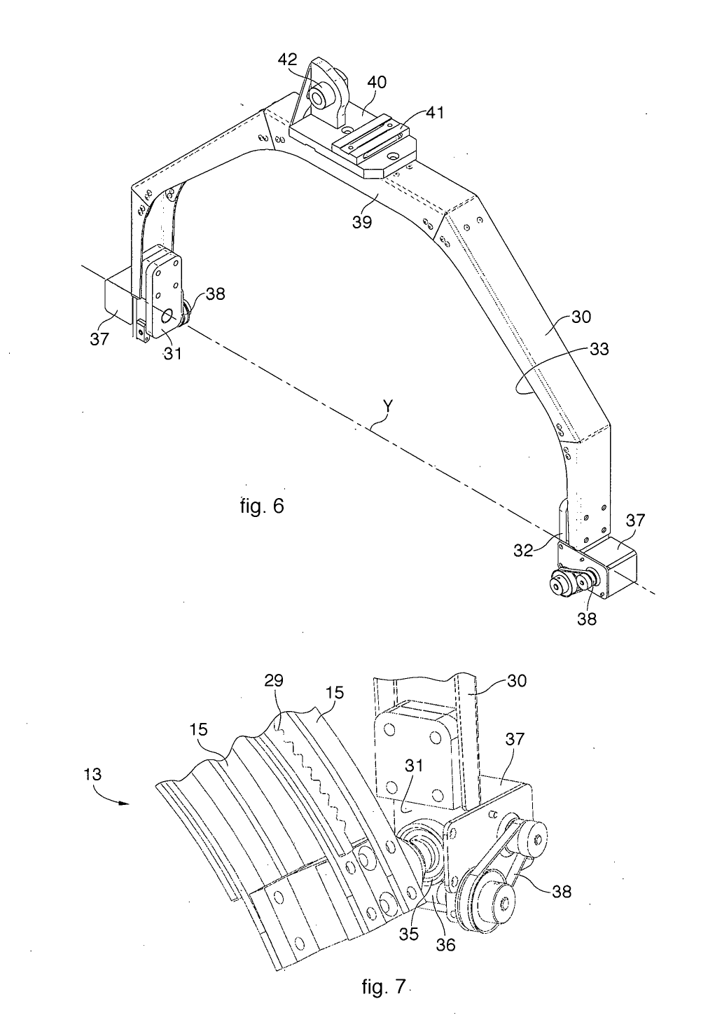

The movement means 12 also comprise an arched support 30 (FIG. 6) with two bases 31 and 32 through which the axis of rotation Y passes. The arched support 30 is shaped so as to define a cavity 33 with sufficiently large sizes, so as to allow the arched guide 13 to rotate liberally inside it around the axis of rotation Y, without interfering with the arched support 30.

The arched guide 13 is pivoted on the bases 31 and 32 of the arched support 30 by means of two pins 34 (FIGS. 3 and 10), coaxial to the axis of rotation Y and each rigidly coupled to a corresponding toothed wheel 35.

Each toothed wheel 35 (FIG. 7) is engaged with a worm screw 36, the rotation of which is commanded by a corresponding third electric motor 37 by means of a return belt 38. In this way the two electric motors 37 are able to perform the selective rotation of the arched guide 13, and therefore of the emitter of radiations 11, around the axis of rotation Y. It must be noted that the connection between each worm screw 36 and the corresponding toothed wheel 35 is also the irreversible type, so that the rotation of the arched guide 13 along the path P2 can occur only by simultaneously driving the electric motors 37.

According to a simplified variant, not shown in the drawings, a single electric motor 37 could be provided to perform the rotation of the arched guide 13 around the axis of rotation Y.

On one upper and central part 39 (FIG. 6) of the arched support 30 a sliding slider 40 is attached, provided with a block 41 and a threaded screw 42.

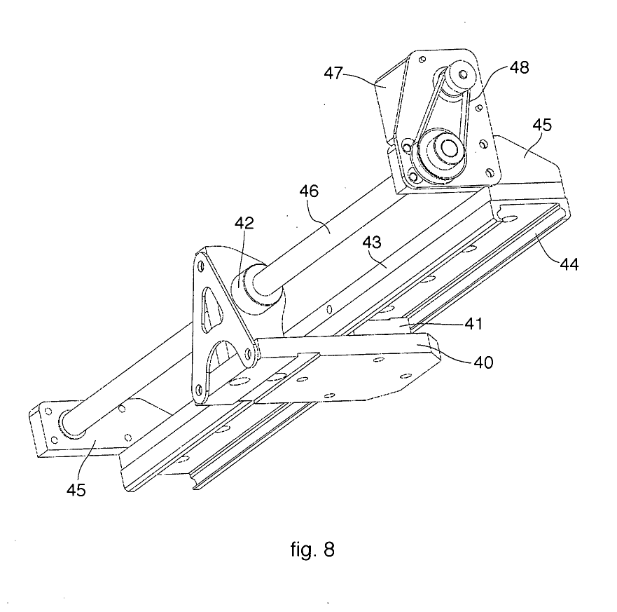

The movement means 12 of the emitter of radiations 11 also comprise a fixed support 43 (FIG. 8) lying on a plane substantially parallel to the base plane PB (therefore substantially horizontal when used in association to a bed as in FIG. 11, or suitably inclined when applied in association to a chair as in FIG. 12) and provided with a rectilinear guide 44, parallel to the axis of displacement X and in which the block 41 of the sliding slider 40 freely slides. The fixed support 43 is also provided with two lateral fins 45 which rotatably support a worm screw 46, screwed into the corresponding threaded screw 42 of the sliding slider 40.

The worm screw 46 is connected to a fourth electric motor 47 by means of a return belt 48. The connection between the worm screw 46 and the corresponding threaded screw 42 is also the irreversible type, so that the movement of the sliding slider 40, and therefore of the arched support 30 along the first path P1, can only occur by driving the electric motor 47.

The fixed support 43 can be connected in an adjustable manner to a pedestal 49 which allows the correct positioning thereof with respect to the patient 50 in relation to the conditions of application.

The apparatus as described heretofore functions as follows.

Each spherical and/or hemispherical surface of the patient to be treated, who is normally lying on the base plane PB along the axis of displacement X, is covered by the apparatus 10 moving the support slider 16 of the emitter of radiations 11 along the meridians of said surface.

The meridians are arcs of a circle and are identified by the intersection of the surface to be treated with the lying plane PG2 of the axis of emission Z of the emitter of radiations 11: the axis of rotation Y of the arched guide 13 is also located on the lying plane PG2.

The surface is therefore completely covered by the combination of the movement of rotation of the arched guide 13 around the axis of rotation Y, which sequentially identifies the meridians, and the sliding along the third path P3, shaped as an arc of a circle with the center in the isocenter O, of the support slider 16 on the arched guide 13.

The movement of the emitter of radiations 11 along the third path P3 occurs by means of the controlled drive of the second electric motor 25, which obtains the movement of the corresponding support slider 16 up to 90° in a clockwise or anti-clockwise direction with respect to an initial position, shown in FIG. 1, in which the axis X1 of the pin 18 is parallel to the axis of displacement X.

Any possible distance between the center of the surface to be treated and the axis of rotation Y of the arched guide 13 and thus the corresponding translation between the meridians and the isocenter O would cause a lack of perpendicularity between the beam of the radiations emitted by the emitter of radiations 11 and the surface to be treated. For this reason the adjustment device 19 has been provided, which is able to carry out a further movement of rotation around the axis X1 of the pin 18, which allows to compensate this effect, at least within certain limits that can be defined in the design phase, for example 60 degrees.

Moreover, the surfaces to be treated of a cylindrical shape are located so that their axis is parallel to the axis of displacement X. In this way the cover of the surfaces is obtained with the combination of the movement of the support slider 16 on the arched guide 13 with the translation of the arched support 30 and of the arched guide 13 along the axis of displacement X.

The rectilinear movement of the emitter of radiations 11 along the first path P1, parallel to the axis of displacement X, is obtained by driving the fourth electric motor 47 in a controlled manner.

To obtain the perpendicularity of this type of scanning it is sufficient that the lying plane of the axis of emission Z of the emitter of radiations 11 is perpendicular to the axis of displacement X.

The possible distance between the axis of the cylindrical surface to be treated and the isocenter O would cause a lack of perpendicularity between the beam of radiations and the surface to be treated. In this case too, the movement of rotation around the axis X1 of the pin 18 of the emitter of radiations 11, mounted on the support slider 16, allows to compensate this effect, at least within certain limits that can be defined in the design phase, as described above.

The movement of the emitter of radiations 11 along the second path P2, also shaped as the arc of a circle, occurs by means of the controlled and simultaneous drive of the two third electric motors 37, which obtain the rotation of the arched guide 13 with respect to the arched support 30, up to 90°, in a clockwise or anti-clockwise direction, with respect to an initial position, shown in FIG. 1.

It is clear therefore that by means of the movement means 12 the emitter of radiations 11, commanded by one or more of the electric motors 25, 37 and 47 is able to position itself along any point of a plurality of hemispherical caps defined by the combination of the three paths P1, P2 and P3, in which each hemispherical cap is defined by the combination of the two paths P2 and P3 shaped as the arc of a circle with the center in the isocenter O.

The patient's flat surfaces to be treated are located so as to be parallel to the base plane PB. In this way the cover of such areas is obtained with the combination of the movement of the support slider 16 on the arched guide 13 and the translation of the arched support 30 and of the arched guide 13 along the axis of displacement X. The rectilinear movement of the emitter of radiations 11 along the first path P1, parallel to the axis of displacement X, is obtained by driving the fourth electric motor 47 in a controlled manner.

To obtain the perpendicularity of this type of scanning, the rotation movement of the emitter of radiations 11 around the axis X1 of the pin 18, mounted on the support slider 16, by means of the first motor 20, is suitably controlled.

It is clear that modifications and/or additions of parts may be made to the apparatus 10 to emit therapeutic radiations as described heretofore, without departing from the field and scope of the present invention.

It is also clear that, although the present invention has been described with reference to some specific examples, a person of skill in the art shall certainly be able to achieve many other equivalent forms of apparatus to emit therapeutic radiations, having the characteristics as set forth in the claims and hence all coming within the field of protection defined thereby.

1. An apparatus to emit therapeutic radiations comprising at least an emitter of radiations configured to emit radiations toward a base plane containing an axis of displacement, movement means configured to selectively move said emitter of radiations both along a second path, arched and lying on a first lying plane passing through said axis of displacement, and also along a third path arched and lying on a second lying plane passing through an axis of rotation, perpendicular to said axis of displacement, wherein said movement means are configured to move said emitter of radiations also along a first path substantially rectilinear and parallel to said axis of displacement, so as to move said emitter of radiations parallel to said base plane, and said movement means comprise:
first guide means, substantially arched in shape, configured to allow said emitter of radiations to move along said third path;
support means configured to support said first guide means so that they can rotate around said axis of rotation; and
second guide means, disposed parallel to said axis of displacement, configured to slidingly support said support means and to allow both said support means and said first guide means and also said emitter of radiations to move together along said first path parallel to said base plane.

2. The apparatus as in claim 1, wherein at least one of said first path and second path are an arc of a circle having as its center a common point.

3. The apparatus as in claim 1, wherein said first guide means comprise an arched guide provided with arched tracks on which a support slider is able to slide, on which slider said emitter of radiations is mounted.

4. The apparatus as in claim 3, wherein on said support slider an adjustment device is mounted, suitable to adjust the position of the axis of emission of said emitter of radiations, comprising a first electric motor able to make said emitter of radiations selectively rotate with respect to said support slider.

5. The apparatus as in claim 3, wherein said support slider is mobile along said arched tracks by means of a second electric motor connected to at least a toothed wheel engaging on a corresponding arched rack of said arched guide.

6. The apparatus as in claim 5, wherein on each lateral flank of said support slider at least four wheels are rotatably mounted, which are disposed in such a manner that at least two of them are in contact with a corresponding lower track of said arched tracks, while at least another two of said wheels are in contact with a corresponding upper track of said arched tracks.

7. The apparatus as in claim 1, wherein said support means comprise a support member, arched in shape, having two bases through which said axis of rotation passes, and defining a cavity having sizes such as to allow said first guide means to rotate inside it around said axis of rotation.

8. The apparatus as in claim 7, wherein said first guide means are suitable to rotate around said axis of rotation, inside said cavity, by means of at least a third electric motor.

9. The apparatus as in claim 7, wherein said second guide means comprise a rectilinear fixed guide, parallel to said first path and in which a sliding slider is able to slide, solid with said support member.

10. The apparatus as in claim 9, wherein said sliding slider is commanded by a fourth electric motor, by means of a worm screw cooperating with a threaded screw of said sliding slider.

11. The apparatus as in claim 1, wherein each movement of said emitter of radiations along any one whatsoever of said three paths is commanded by at least one electric motor, by means of mechanical members which guarantee the motion is irreversible.
